(19) United States
(12) Patent Application Publication
(10) Pub. No.: US 2021/0113790 A1
(43) Pub. Date: Apr. 22, 2021

(54) REUSABLE TRAINING VALVE ASSEMBLY WELL-SUITED FOR USE WITH A CARDIOPULMONARY RESUSCITATION (CPR) TRAINING MASK OR WITH A CPR MASK

(71) Applicant: Wayne Eddy, Hopkinton, MA (US)

(72) Inventor: Wayne Eddy, Hopkinton, MA (US)

(21) Appl. No.: 17/006,816

(22) Filed: Aug. 29, 2020

Related U.S. Application Data
(60) Provisional application No. 62/893,555, filed on Aug. 29, 2019.

(51) Int. Cl. A61M 16/00; G09B 23/28; A61M 16/10; A61M 16/20

(52) U.S. Cl. CPC A61M 16/0048; A61M 16/208; A61M 16/105; G09B 23/288

(57) ABSTRACT

A cardiopulmonary resuscitation (CPR) training valve assembly well-suited for use with a CPR training mask or CPR mask. In one embodiment, the assembly includes a first housing member, a second housing member, and an air filter. The first housing member may be a tubular structure having a proximal end and a distal end, the proximal end being dimensioned for insertion into a user's mouth. The second housing member may be a tubular structure having a proximal end and a distal end, the distal end being dimensioned for insertion into, for example, a port of a CPR training mask. The second housing member and the first housing member may be repeatedly coupled to and uncoupled from one another. The air filter is disposed within one of the first and second housing members and is positioned to be removable therefrom when the first and second housing members are uncoupled from one another.

### CROSS-REFERENCE TO RELATED APPLICATIONS

The present application claims the benefit under 35 U.S.C. 119(e) of U.S. Provisional Patent Application No. 62/893,555, inventor Wayne Eddy, filed Aug. 29, 2019, the disclosure of which is incorporated herein by reference.

### BACKGROUND OF THE INVENTION

The present invention relates generally to cardiopulmonary resuscitation and relates more particularly to cardiopulmonary resuscitation training devices.

Cardiopulmonary resuscitation (commonly referred to as “CPR”) is an emergency medical technique that is useful in many situations in which a patient's breathing and/or heartbeat has stopped. CPR typically involves having a rescuer apply chest compressions to a patient in an effort to restore some degree of blood circulation to the patient. In addition to applying chest compressions, the rescuer may also provide artificial ventilation to the patient by exhaling into the patient's mouth and/or nose (“mouth-to-mouth resuscitation”). As can readily be appreciated, it can be unhygienic for a rescuer to place his mouth directly on a patient's mouth during the performance of mouth-to-mouth resuscitation. This is particularly so since it is typical for a patient to cough and/or to regurgitate once rescued. Consequently, it is not uncommon for trained medical personnel to place a CPR mask with a valve assembly over a patient's face during the performance of mouth-to-mouth resuscitation. The CPR mask includes a generally bulbous structure that typically covers the patient's mouth and nose. A bladder is positioned at the base of the bulbous structure and forms a substantially airtight seal around the patient's mouth and nose. The valve assembly comprises a generally tubular housing, one end of which is insertable into a port in the mask and the other end of which is insertable into the rescuer's mouth. The valve assembly further includes a one-way valve disposed within the tubular housing. The one-way valve enables a rescuer to exhale into the mask while, at the same time, preventing fluids and solids from passing from the patient to the rescuer.

Because CPR is recognized as a valuable lifesaving technique, there is a clear need for medical personnel to become trained in performing CPR and to practice performing CPR periodically thereafter. Such training and practice are typically performed on manikins that have been specifically designed for this purpose. In a similar vein, CPR training masks and CPR training valve assemblies that are intended for use with such manikins have also been devised. A CPR training mask of the type intended for use with a manikin is generally similar to a CPR mask of the type used on an actual patient, with one difference being that the CPR training mask typically does not include a strap for securing the mask around the head of the manikin. A CPR training valve assembly of the type intended for use with a manikin typically differs from a CPR valve assembly of the type used on actual patient in that the CPR training valve assembly does not typically include a one-way valve, but rather, includes a microporous air filter or other barrier material to minimize contamination of the manikin by a user. (Despite the fact that a CPR training valve assembly of the type described above typically does not actually include a valve, the convention in the industry is to identify such an assembly as a training valve or CPR training valve.) An example of a CPR training mask of the type described above is commercially available from WNL Products as PRACTI-MASK® CPR training mask (Work 'N Leisure Products, Inc., Holliston, Mass.), and an example of a CPR training valve assembly of the type described above is commercially available from WNL Products as PRACTI-VALVE® CPR training valve (Work 'N Leisure Products, Inc., Holliston, Mass.).

Referring now to FIGS. 1 through 4, there are shown various views of a conventional CPR training valve assembly, the aforementioned conventional CPR training valve assembly being represented generally by reference numeral 11.

CPR training valve assembly 11, which corresponds generally to the aforementioned PRACTI-VALVE® CPR training valve and which is intended for use with a CPR training mask, such as the aforementioned PRACTI-MASK® CPR training mask (but may also be used with a CPR mask of the type used on an actual patient), comprises a first housing member 13, a second housing member 15, a third housing member 17, and a barrier 19.

First housing member 13, which is a one-piece structure made of a rigid, molded polymer, comprises a main portion 21 and a flange 23. Main portion 21 is generally cylindrical and tubular in shape and includes a side wall 25 terminating at an open proximal end 27 and an open distal end 29. An annular recess 31 is provided along the interior surface of side wall 25 a short distance from open proximal end 27, annular recess 31 being dimensioned to receive the peripheral edge of barrier 19 therewithin. Side wall 25 has a generally uniform outer diameter over its length. The inner diameter of side wall 25 is generally uniform from open distal end 29 to annular recess 31 (not including annular recess 31, which has an increased inner diameter relative to the portion of side wall 25 distal thereto), and the inner diameter of side wall 25 is generally uniform from open proximal end 27 to annular recess 31 (not including annular recess 31, which has an increased inner diameter relative to the portion of side wall 25 proximal thereto). The inner diameter of side wall 25 is slightly reduced on the proximal side of annular recess 31 as compared to the inner diameter of side wall 25 on the distal side of annular recess 31.

Flange 23 is a generally annular structure that extends radially outwardly from the exterior surface of main portion 21 a short distance from open distal end 29. Flange 23 is shaped to enable a user to more easily grip training valve assembly 11, thereby facilitating the removal of training valve assembly 11 from the port of a CPR training mask or from the port of a CPR mask of the type used on an actual patient.

Second housing member 15 is a tubular, one-piece structure made of a rigid, molded polymer. Second housing member 15 includes a side wall 35 terminating at an open proximal end 37 and an open distal end 39. Second housing member 15 is partially coaxially inserted into first housing member 13 and is sonically welded permanently thereto, with open proximal end 37 of second housing member 15 being situated within main portion 21 of first housing member 13 and with open distal end 39 of second housing member 15 being positioned distally relative to open distal end 29 of first housing member 13. Side wall 35 of second housing member 15 is shaped to include a proximal portion 36-1 that is generally cylindrical and is dimensioned for insertion into first housing member 13 and a distal portion 36-2 that is shaped to taper slightly inwardly towards open distal end 39 to facilitate second housing member 15 being inserted into and removed from the port of a CPR training mask or from the port of a CPR mask of the type used on an actual patient.

Third housing member 17, which is a one-piece structure made of a rigid, molded polymer, is shaped to include a porous grid 41 defined by intersecting bars joined to an annular rim 43. Porous grid 41 is designed to permit a user to freely exhale therethrough and to provide some form of restraint to materials, including barrier 19, from passing proximally therethrough. Third housing member 17, which is dimensioned so that rim 43 generally matches the size of side wall 25 at open proximal end 27, is sonically welded permanently to first housing member 15 over open proximal end 27.

Barrier 19 is a one-piece structure made of a microporous air filter material. Barrier 19 is generally disc-shaped and is dimensioned so that its peripheral edge is received within annular recess 31 of first housing member 13 in such a way that barrier 19 extends transversely across the lumen of main portion 21. Barrier 19 serves to minimize the transmission of bacteria and other contaminants from a user to the manikin.

In use, training valve assembly 11 is inserted into a port of a CPR training mask or into a port of a CPR mask of the type used on an actual patient (see FIG. 5, which shows training valve assembly 11 inserted into a port 51 of a CPR training mask 53), and the CPR training mask or CPR mask of the type used on an actual patient is positioned over the face of a manikin. The user then practices mouth-to-mouth resuscitation on the manikin by inserting the proximal end of training valve assembly 11 into the user's mouth and then exhaling into training valve assembly 11 according to established CPR procedures. (Depending on the type of manikin being used, the user may also perform chest compressions on the manikin.)

Training valve assembly 11 is a single-use device. Accordingly, after a user has practiced performing mouth-to-mouth resuscitation on the manikin, the user removes training valve assembly 11 from the CPR training mask or CPR mask and properly disposes of training valve 11 in its entirety. The CPR training mask or CPR mask is then ready to receive a new training valve assembly 11. (Preferably, the CPR training mask or CPR mask is washed between uses.)

### SUMMARY OF THE INVENTION

The present inventor has recognized that a CPR training valve assembly that is a single-use device results in higher costs and generates more waste than a comparable CPR training valve assembly that includes at least some reusable components.

Accordingly, it is an object of the present invention to provide a CPR training valve assembly that includes at least some components that are reusable.

Therefore, according to one aspect of the invention, there is provided a training valve assembly well-suited for use with a cardiopulmonary resuscitation training mask or a cardiopulmonary resuscitation mask, the training valve assembly comprising (a) a first housing member; (b) a second housing member, the second housing member being coupled to the first housing member in such a way as to permit the first housing member and the second housing member to be uncoupled from one another and then recoupled to one another; and (c) an air filter disposed within at least one of the first housing member and the second housing member and positioned so as to be removable therefrom when the first housing member and the second housing member are uncoupled from one another so as to permit replacement of the air filter.

In a more detailed feature of the invention, the first housing member may comprise a tubular structure having a proximal end and a distal end, and the proximal end may be dimensioned for insertion into a mouth of a user.

In a more detailed feature of the invention, the first housing member may comprise a proximal portion and a distal portion, the proximal portion may comprise a first tubular structure, the distal portion may comprise a second tubular structure, the first tubular structure may have a first lumen, the second tubular structure may have a second lumen, and the first and second lumens may be coaxial and in fluid communication with one another.

In a more detailed feature of the invention, each of the first tubular structure of the first housing member and the second tubular structure of the first housing member may have an inner diameter and an outer diameter, and the inner diameter and the outer diameter of the second tubular structure of the first housing member may be reduced relative to that of the first tubular structure of the first housing member.

In a more detailed feature of the invention, the proximal portion of the first housing member may comprise a proximal end, and the proximal end may comprise a porous grid.

In a more detailed feature of the invention, the distal portion of the first housing member may be externally threaded.

In a more detailed feature of the invention, the first housing member may further comprise an annular flange extending radially outwardly between the proximal portion and the distal portion.

In a more detailed feature of the invention, the second housing member may comprise a tubular structure having a proximal end and a distal end, and the distal end may be dimensioned for insertion into a port of a cardiopulmonary resuscitation training mask or a cardiopulmonary resuscitation mask.

In a more detailed feature of the invention, the second housing member may comprise a proximal portion and a distal portion, the proximal portion may comprise a first tubular structure, the distal portion may comprise a second tubular structure, the first tubular structure may have a first lumen, the second tubular structure may have a second lumen, and the first and second lumens may be coaxial and in fluid communication with one another.

In a more detailed feature of the invention, each of the first tubular structure of the second housing member and the second tubular structure of the second housing member may have an inner diameter, the inner diameter of the second tubular structure of the second housing member may be reduced relative to that of the first tubular structure of the second housing member, an annular shelf may be formed at a juncture of the first tubular structure of the second housing member and the second tubular structure of the second housing member, and the air filter may be mounted on the annular shelf.

In a more detailed feature of the invention, the first tubular structure of the second housing member may comprise a side wall that may be generally cylindrical, and the second tubular structure of the second housing member may comprise a side wall that may be generally frustoconical.

In a more detailed feature of the invention, the proximal portion of the second housing member may be internally threaded.

In a more detailed feature of the invention, the first housing member and the second housing member may comprise mating threads to permit the first housing member and the second housing member to be screwed to one another and unscrewed from one another one or more times.

In a more detailed feature of the invention, the training valve assembly may further comprise a ring, and the ring may be coupled to the air filter.

According to another aspect of the invention, there is provided a training valve assembly well-suited for use with a cardiopulmonary resuscitation training mask or a cardiopulmonary resuscitation mask, the training valve assembly comprising (a) a first housing member, wherein the first housing member comprises a proximal portion and a distal portion, the proximal portion comprising a first tubular structure, the distal portion comprising a second tubular structure, the first tubular structure having a first lumen, the second tubular structure having a second lumen, the first and second lumens being coaxial and in fluid communication with one another, the proximal portion comprising a proximal end dimensioned for insertion into a mouth of a user, the distal portion being externally threaded; (b) a second housing member, the second housing member being coupled to the first housing member in such a way as to permit the first housing member and the second housing member to be uncoupled from one another and then recoupled to one another, wherein the second housing member comprises a proximal portion and a distal portion, the proximal portion comprising a first tubular structure, the distal portion comprising a second tubular structure, the first tubular structure having a first lumen, the second tubular structure having a second lumen, the first and second lumens being coaxial and in fluid communication with one another, an annular shelf being formed at a juncture of the first and second lumens of the second housing member, the proximal portion of the second housing member being internally threaded and dimensioned to mate with the distal portion of the first housing member; and (c) an air filter mounted on the annular shelf and removable from the second housing member when the first housing member and the second housing member are uncoupled from one another.

According to still another aspect of the invention, there is provided a method comprising (a) providing the training valve assembly as described above; (b) uncoupling the first housing member and the second housing member; (c) replacing the air filter with a new air filter; and (d) recoupling the first housing member and the second housing member.

In a more detailed feature of the invention, the first housing member and the second housing member may comprise mating threads, and said uncoupling and recoupling steps may be performed by unscrewing and screwing together the first housing member and the second housing member using said mating threads.

According to a further aspect of the invention, there is provided a training valve assembly kit for use with a cardiopulmonary resuscitation training mask or a cardiopulmonary resuscitation mask, the training valve assembly kit comprising (a) a first housing member; (b) a second housing member, the second housing member being coupled to the first housing member in such a way as to permit the first housing member and the second housing member to be uncoupled from one another and then recoupled to one another; and (c) a plurality of interchangeable barrier units, wherein each of the plurality of interchangeable barrier units is adapted to be disposed, one at a time, within at least one of the first housing member and the second housing member and removable therefrom when the first housing member and the second housing member are uncoupled from one another so as to permit replacement of an installed barrier unit.

In a more detailed feature of the invention, each of the plurality of interchangeable barrier units may comprise an air filter.

Additional objects, as well as aspects, features and advantages, of the present invention will be set forth in part in the description which follows, and in part will be obvious from the description or may be learned by practice of the invention. In the description, reference is made to the accompanying drawings which form a part thereof and in which is shown by way of illustration various embodiments for practicing the invention. The embodiments will be described in sufficient detail to enable those skilled in the art to practice the invention, and it is to be understood that other embodiments may be utilized and that structural changes may be made without departing from the scope of the invention. The following detailed description is, therefore, not to be taken in a limiting sense, and the scope of the present invention is best defined by the appended claims.

### BRIEF DESCRIPTION OF THE DRAWINGS

The accompanying drawings, which are hereby incorporated into and constitute a part of this specification, illustrate various embodiments of the invention and, together with the description, serve to explain the principles of the invention. These drawings are not necessarily drawn to scale, and certain components may have undersized and/or oversized dimensions for purposes of explication or may omit certain features for purposes of clarity. In the drawings wherein like reference numeral represent like parts:

FIG. 1 is a proximal perspective view of a conventional CPR training valve assembly;

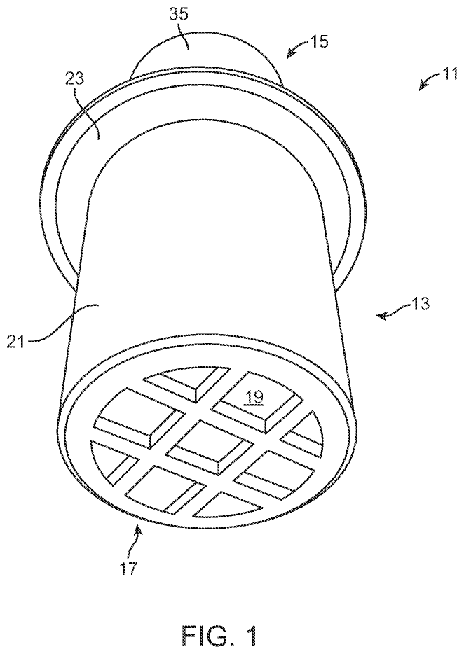

FIG. 2 is a distal perspective view of the conventional CPR training valve assembly shown in FIG. 1;

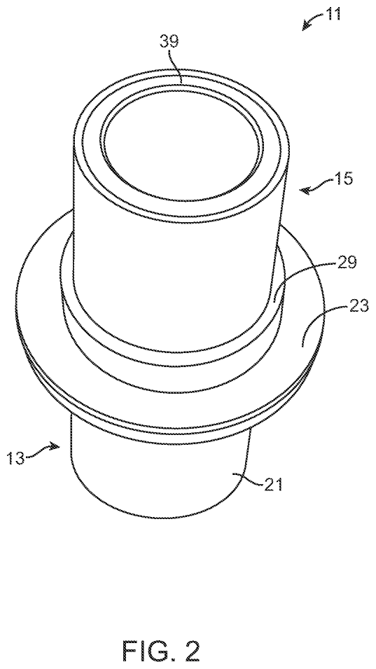

FIG. 3 is an exploded perspective view of the conventional CPR training valve assembly shown in FIG. 1;

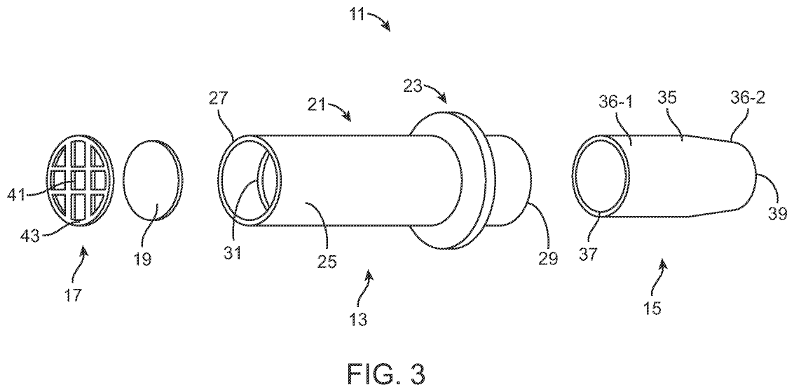

FIG. 4 is a longitudinal section view of the conventional CPR training valve assembly shown in FIG. 1;

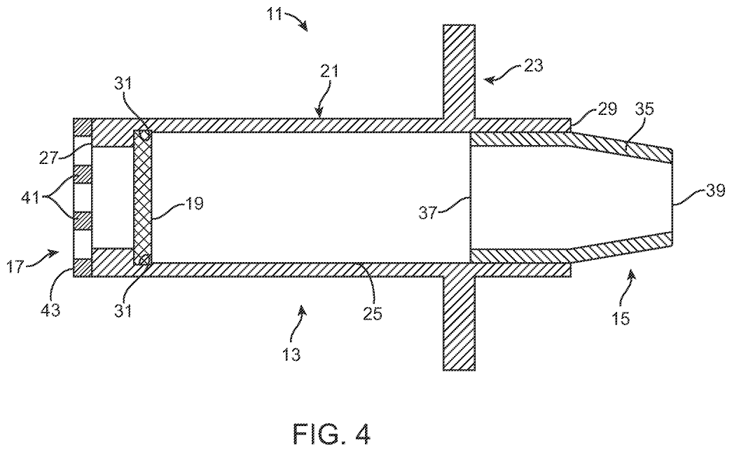

FIG. 5 is a perspective view of the conventional CPR training valve assembly of FIG. 1 inserted into the port of a conventional CPR training mask;

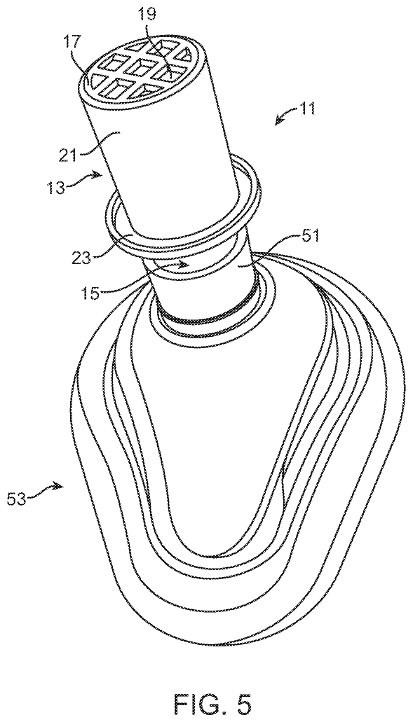

FIG. 6 is a proximal perspective view of one embodiment of a CPR training valve assembly constructed according to the teachings of the present invention;

FIG. 7 is an exploded perspective view of the CPR training valve assembly shown in FIG. 6;

FIGS. 8(a) and 8(b) are side and longitudinal section views, respectively, of the first housing member shown in FIG. 6;

FIGS. 8(c) and 8(d) are side and proximal end views, respectively, of the first housing member shown in FIG. 6, said side and proximal end views including exemplary dimensions;

FIGS. 9(a) and 9(b) are proximal end and longitudinal section views, respectively, of the second housing member shown in FIG. 6;

FIG. 9(c) is a longitudinal section view of the second housing member shown in FIG. 6, said longitudinal section view including exemplary dimensions; and

FIG. 10 is a perspective view of one embodiment of a kit for use in making a CPR training valve assembly, the kit being constructed according to the teachings of the present invention.

### DETAILED DESCRIPTION OF THE INVENTION

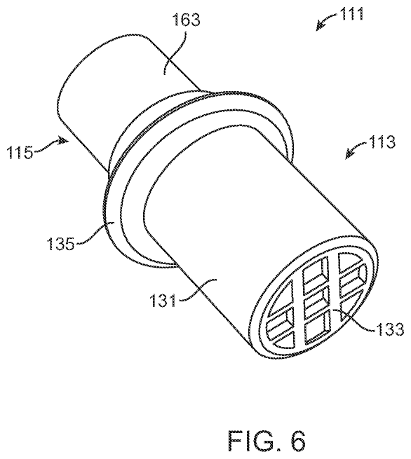

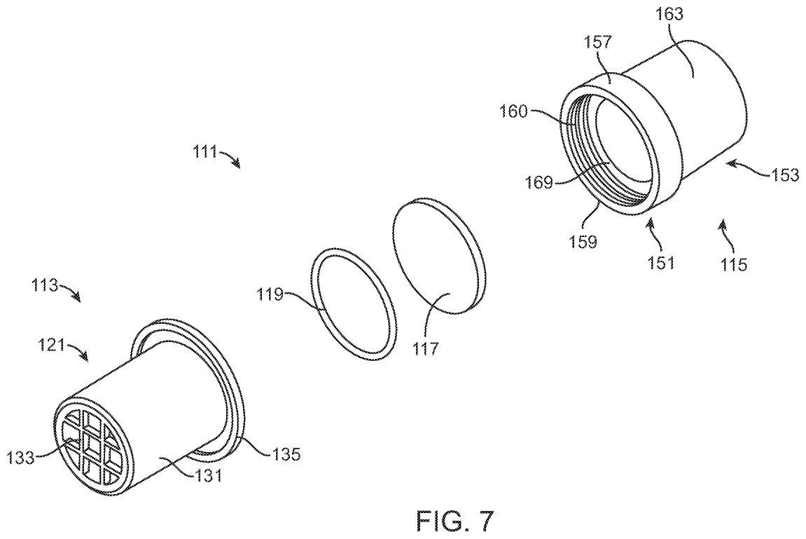

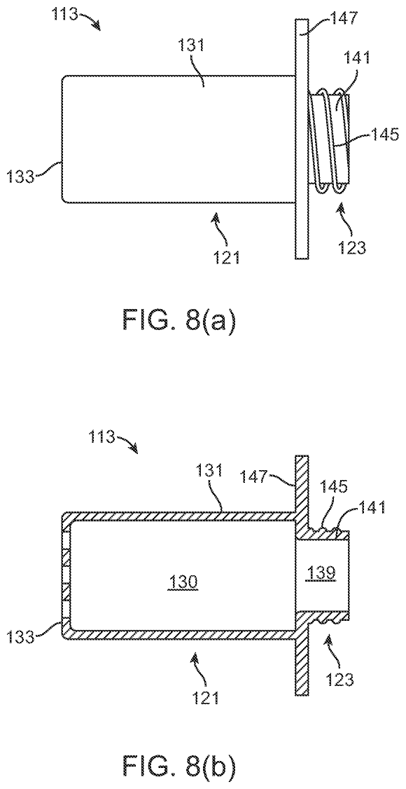

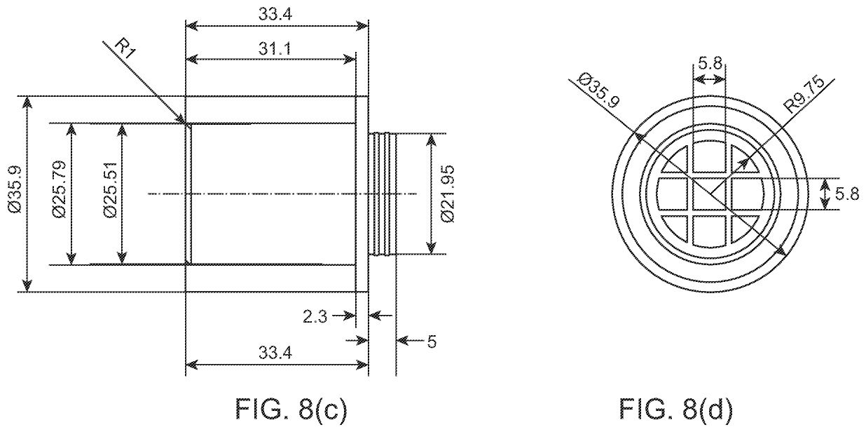

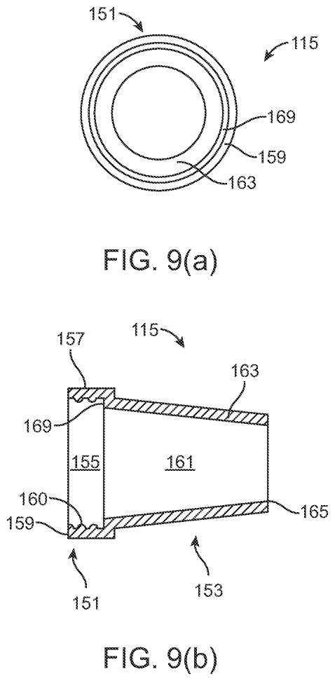

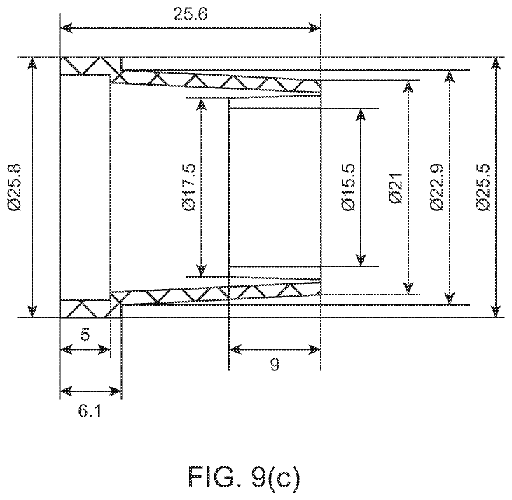

Referring now to FIGS. 6 and 7, there are shown various views of one embodiment of a CPR training valve assembly constructed according to the teachings of the present invention, the CPR training valve assembly being represented generally by reference numeral 111. Details of CPR training valve assembly 111 that are discussed elsewhere in this application or that are not critical to an understanding of the invention may be omitted from one or more of FIGS. 6 and 7 (and/or one or more of FIGS. 8(a)-8(d) and 9(a)-9(c)) and/or from the accompanying description herein or may be shown in one or more of FIGS. 6 and 7 (and/or one or more of FIGS. 8(a)-8(d) and 9(a)-9(c)) and/or described herein in a simplified manner.

CPR training valve assembly 111 may comprise a first housing member 113, a second housing member 115, a barrier 117, and a ring 119.

First housing member 113, which is also shown separately in FIGS. 8(a) and 8(b), may be a one-piece structure made of a rigid, molded polymer. First housing member 113 may comprise a proximal portion 121 and a distal portion 123. Proximal portion 121, which may be a generally tubular structure having an axial lumen 130, may be shaped to include a side wall 131, which may be generally cylindrical, and a proximal end 133, which may be shaped to include a porous grid defined by a plurality of intersecting bars. Said porous grid may function analogously to porous grid 41 of training valve assembly 11.

Distal portion 123 of first housing member 113 may be a generally tubular structure having an axial lumen 139. Distal portion 123 may be shaped to include a side wall 141, which may be generally cylindrical. Lumen 139 of distal portion 123 and lumen 130 of proximal portion 121 may be coaxial and in fluid communication with one another; however, side wall 141 of distal portion 123 may be of reduced inner and outer diameter relative to side wall 131 of proximal portion 121. For reasons to become apparent below, distal portion 123 may be shaped to include at least one external thread 145. An annular flange 147 may extend radially outwardly at the juncture of proximal portion 121 and distal portion 123. Flange 147 may function analogously to flange 23 of CPR training valve assembly 11.

FIGS. 8(c) and 8(d) are additional views of first housing member 113 and include some exemplary dimensions (in millimeters). Such dimensions are merely illustrative and are not intended to be limiting.

Second housing member 115, which is also shown separately in FIGS. 9(a) and 9(b), may be a one-piece structure made of a rigid, molded polymer. Second housing member 115 may comprise a proximal portion 151 and a distal portion 153. Proximal portion 151, which may be a generally tubular structure having an axial lumen 155, may be shaped to include a side wall 157, which may be generally cylindrical, and an open proximal end 159. Side wall 157 of proximal portion 151 may be appropriately dimensioned to coaxially receive distal portion 123 of first housing member 115 and may include at least one internal thread 160 for mating engagement with external thread 145 of distal portion 123. In this fashion, first housing member 113 and second housing member 115 may be screwed together and unscrewed from one another, as desired.

Distal portion 153 of second housing member 115, which may be a generally tubular structure having an axial lumen 161, may be shaped to include a side wall 163, which may be generally frustoconical (i.e., tapering inwardly distally), and an open distal end 165. Lumen 155 of proximal portion 151 and lumen 161 of distal portion 153 may be coaxial and in fluid communication with one another; however, side wall 163 of distal portion 153 may be of reduced inner and outer diameter relative to side wall 157 of proximal portion 151. As a result, an annular shelf 169 may be formed at the juncture of proximal portion 151 and distal portion 153.

FIG. 9(c) is an additional view of second housing member 115 and includes some exemplary dimensions (in millimeters). Such dimensions are merely illustrative and are not intended to be limiting. Internal thread 160 is not shown in FIG. 9(c).

Barrier 117, which may be a one-piece structure made of a microporous air filter material, may be similar in composition to barrier 19 of CPR training valve assembly 11. An example of a microporous air filter material suitable for use as barrier 117 may be a non-woven polypropylene, such as a 25 g/m² non-woven polypropylene of the type commercially available as TDK-25 filter material from Tianjin Teda Co. Ltd., Tianjin, China. Barrier 117 may be appropriately dimensioned so that its peripheral edge may be seated upon annular shelf 169 of second housing member 115, whereby barrier 117 may cover the proximal end of axial lumen 161. As can be appreciated, the placement of barrier 117 in training valve assembly 111 is more distal than the placement of barrier 19 in training valve assembly 11.

Ring 119, which may be made of plastic or the like, may be used to provide support to barrier 117 and may be seated on top of (i.e., proximal to) barrier 117 or between annular shelf 169 and barrier 117. An adhesive (not shown) may be used to adhere, temporarily or permanently, ring 119 to barrier 117.

Where first housing member 113 has the dimensions provided in FIGS. 8(c) and 8(d) and where second housing member 115 has the dimensions provided in FIG. 9(c), barrier 117 may be a circular member having a diameter of 21 mm, and ring 119 may be an annular member having an outer diameter of 21 mm and an inner diameter of 16 mm.

To assemble training valve assembly 111, barrier 117 and ring 119 may be seated on shelf 169, and first housing member 113 may be screwed into second housing member 115.

In use, distal end 165 of second housing member 115 may be inserted into the port of a CPR training mask (or into the port of a CPR mask of the type used on an actual patient or other similar type of mask), and a user may exhale into proximal end 133 of first housing member 113. After use, CPR training valve assembly 111 may be removed from the CPR training mask (or other mask), first housing member 113 may be unscrewed from second housing member 115, and barrier 117 and ring 119 may be removed from second housing member 113 and disposed of properly. First housing member 113 and second housing member 115 then may be cleaned, for example, by soaking first housing member 113 and second housing member 115 for 10 minutes in a solution of 1 part bleach and 64 parts water, then rinsing first housing member 113 and second housing member 115 with water, and then allowing first housing member 113 and second housing member 115 to dry. Then, a new barrier 117 and a new ring 119 may be inserted into second housing member 115 for seating on shelf 169 as described above, and first housing member 113 may be screwed into second housing member 115. CPR training valve assembly 111 may then be inserted into the port of a CPR training mask or other mask and used as described above. The foregoing steps may be repeated one or more times.

It should be noted that, although both barrier 117 and ring 119 are intended to be replaced as a unit with each use, if barrier 117 and ring 119 are not adhered to one another, one could replace barrier 117 with each use and could simply wash ring 119, instead of replacing ring 119, between uses.

As can be appreciated, one advantage of CPR training valve assembly 111, as compared to CPR training valve assembly 11, is that first housing member 113 and second housing member 115 may be easily separated from one another, for example, by unscrewing, thereby enabling barrier 117 (and ring 119) to be replaced as desired (preferably after each use). As a result, one need not dispose of the entirety of CPR training valve assembly 111 after each use, but rather, may simply replace barrier 117 (and ring 119). This results in a significant reduction in manufacturing and waste, as compared to single-use devices like CPR training valve assembly 11. It should be noted that barrier 19 from CPR training valve assembly 11 cannot be removed from its housing without destroying the housing. This is because first housing member 13 is permanently bonded to each of second housing member 15 and third housing member 17.

Also, it should be noted that, if first housing member 113 or second housing member 115 becomes damaged, it is possible to replace such parts without replacing the entire assembly.

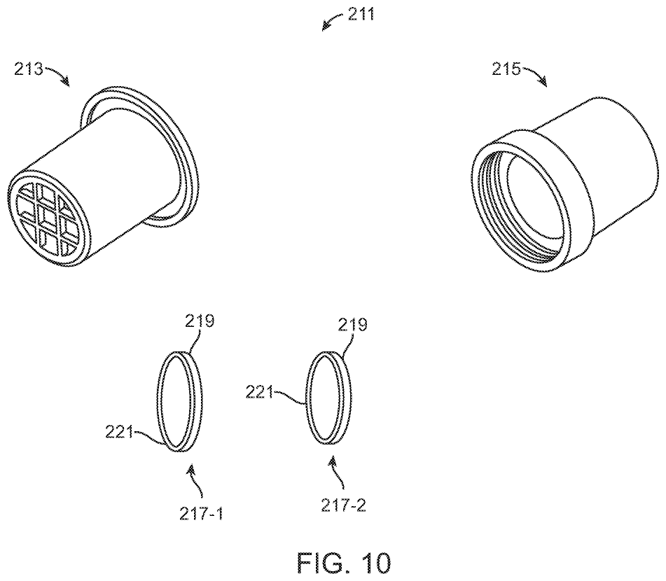

Referring now to FIG. 10, there is shown a perspective view of one embodiment of a CPR training valve assembly kit constructed according to the teachings of the present invention, the CPR training valve assembly kit being represented generally by reference numeral 211.

Kit 211 may comprise a first housing member 213, a second housing member 215, and a plurality of identical barrier units 217-1 and 217-2.

First housing member 213 may be identical to first housing member 113, and second housing member 215 may be identical to second housing member 115. Each of barrier units 217-1 and 217-2 may comprise a barrier 219, which may be identical to barrier 117, and a ring 221, which may be identical to ring 119. Barrier 219 and ring 221 may be permanently bonded to one another with a suitable adhesive (not shown).

In use, barrier unit 217-1 may be placed in second housing member 215, and first housing member 213 and second housing member 215 may be screwed together. The resulting CPR training valve assembly may then be used in the same manner described above for CPR training valve assembly 111. After use, first housing member 213 and second housing member 215 may be unscrewed from one another, barrier unit 217-1 may be removed from second housing member 215 and disposed of properly, first housing member 213 and second housing member 215 may be cleaned, a new barrier unit, e.g., barrier unit 217-2, may be placed in second housing member 215, and first housing member 213 and second housing member 215 may be screwed together once again. The resulting CPR training valve assembly may then be used in the same manner described above for CPR training valve assembly 111.

It should be noted that, although kit 211 is shown in FIG. 10 having two identical barrier units 217-1 and 217-2, kit 211 could have a greater or lesser number of such barrier units. Also, while barrier units 217-1 and 217-2 are preferably identical, they need not be.

The embodiments of the present invention described above are intended to be merely exemplary and those skilled in the art shall be able to make numerous variations and modifications to it without departing from the spirit of the present invention. All such variations and modifications are intended to be within the scope of the present invention as defined in the appended claims.

What is claimed is:

1. A training valve assembly well-suited for use with a cardiopulmonary resuscitation training mask or a cardiopulmonary resuscitation mask, the training valve assembly comprising:
(a) a first housing member;
(b) a second housing member, the second housing member being coupled to the first housing member in such a way as to permit the first housing member and the second housing member to be uncoupled from one another and then recoupled to one another; and
(c) an air filter disposed within at least one of the first housing member and the second housing member and positioned so as to be removable therefrom when the first housing member and the second housing member are uncoupled from one another so as to permit replacement of the air filter.

2. The training valve assembly as claimed in claim 1 wherein the first housing member comprises a tubular structure having a proximal end and a distal end, wherein the proximal end is dimensioned for insertion into a mouth of a user.

3. The training valve assembly as claimed in claim 1 wherein the first housing member comprises a proximal portion and a distal portion, the proximal portion comprising a first tubular structure, the distal portion comprising a second tubular structure, the first tubular structure having a first lumen, the second tubular structure having a second lumen, the first and second lumens being coaxial and in fluid communication with one another.

4. The training valve assembly as claimed in claim 3 wherein each of the first tubular structure of the first housing member and the second tubular structure of the first housing member has an inner diameter and an outer diameter and wherein the inner diameter and the outer diameter of the second tubular structure of the first housing member is reduced relative to that of the first tubular structure of the first housing member.

5. The training valve assembly as claimed in claim 3 wherein the proximal portion of the first housing member comprises a proximal end, wherein the proximal end comprises a porous grid.

6. The training valve assembly as claimed in claim 3 wherein the distal portion of the first housing member is externally threaded.

7. The training valve assembly as claimed in claim 3 wherein the first housing member further comprises an annular flange extending radially outwardly between the proximal portion and the distal portion.

8. The training valve assembly as claimed in claim 1 wherein the second housing member comprises a tubular structure having a proximal end and a distal end, wherein the distal end is dimensioned for insertion into a port of a cardiopulmonary resuscitation training mask or a cardiopulmonary resuscitation mask.

9. The training valve assembly as claimed in claim 1 wherein the second housing member comprises a proximal portion and a distal portion, the proximal portion comprising a first tubular structure, the distal portion comprising a second tubular structure, the first tubular structure having a first lumen, the second tubular structure having a second lumen, the first and second lumens being coaxial and in fluid communication with one another.

10. The training valve assembly as claimed in claim 9 wherein each of the first tubular structure of the second housing member and the second tubular structure of the second housing member has an inner diameter and wherein the inner diameter of the second tubular structure of the second housing member is reduced relative to that of the first tubular structure of the second housing member, wherein an annular shelf is formed at a juncture of the first tubular structure of the second housing member and the second tubular structure of the second housing member, and wherein the air filter is mounted on the annular shelf.

11. The training valve assembly as claimed in claim 10 wherein the first tubular structure of the second housing member comprises a side wall that is generally cylindrical and wherein the second tubular structure of the second housing member comprises a side wall that is generally frustoconical.

12. The training valve assembly as claimed in claim 9 wherein the proximal portion of the second housing member is internally threaded.

13. The training valve assembly as claimed in claim 1 wherein the first housing member and the second housing member comprise mating threads to permit the first housing member and the second housing member to be screwed to one another and unscrewed from one another one or more times.

14. The training valve assembly as claimed in claim 1 further comprising a ring, the ring coupled to the air filter.

15. A training valve assembly well-suited for use with a cardiopulmonary resuscitation training mask or a cardiopulmonary resuscitation mask, the training valve assembly comprising:
(a) a first housing member, wherein the first housing member comprises a proximal portion and a distal portion, the proximal portion comprising a first tubular structure, the distal portion comprising a second tubular structure, the first tubular structure having a first lumen, the second tubular structure having a second lumen, the first and second lumens being coaxial and in fluid communication with one another, the proximal portion comprising a proximal end dimensioned for insertion into a mouth of a user, the distal portion being externally threaded;
(b) a second housing member, the second housing member being coupled to the first housing member in such a way as to permit the first housing member and the second housing member to be uncoupled from one another and then recoupled to one another, wherein the second housing member comprises a proximal portion and a distal portion, the proximal portion comprising a first tubular structure, the distal portion comprising a second tubular structure, the first tubular structure having a first lumen, the second tubular structure having a second lumen, the first and second lumens being coaxial and in fluid communication with one another, an annular shelf being formed at a juncture of the first and second lumens of the second housing member, the proximal portion of the second housing member being internally threaded and dimensioned to mate with the distal portion of the first housing member; and
(c) an air filter mounted on the annular shelf and removable from the second housing member when the first housing member and the second housing member are uncoupled from one another.

16. A method comprising:
(a) providing the training valve assembly of claim 1;
(b) uncoupling the first housing member and the second housing member;
(c) replacing the air filter with a new air filter; and
(d) recoupling the first housing member and the second housing member.

17. The method as claimed in claim 1 wherein the first housing member and the second housing member comprise mating threads and wherein said uncoupling and recoupling steps are performed by unscrewing and screwing together the first housing member and the second housing member using said mating threads.

18. A training valve assembly kit for use with a cardiopulmonary resuscitation training mask or a cardiopulmonary resuscitation mask, the training valve assembly kit comprising:
(a) a first housing member;
(b) a second housing member, the second housing member being coupled to the first housing member in such a way as to permit the first housing member and the second housing member to be uncoupled from one another and then recoupled to one another; and
(c) a plurality of interchangeable barrier units, wherein each of the plurality of interchangeable barrier units is adapted to be disposed, one at a time, within at least one of the first housing member and the second housing member and removable therefrom when the first housing member and the second housing member are uncoupled from one another so as to permit replacement of an installed barrier unit.

19. The training valve assembly kit as claimed in claim 18 wherein each of the plurality of interchangeable barrier units comprises an air filter.
